(19) United States
(12) Patent Application Publication
(10) Pub. No.: US 2026/0355765 A1
(43) Pub. Date: Nov. 12, 2026

(54) DEVICE FOR DISPENSING A FLUID PRODUCT

(71) Applicant: APTAR FRANCE SAS, LE NEUBOURG (FR)

(72) Inventor: Ludovic PETIT, VITOT (FR)

(73) Assignee: APTAR FRANCE SAS, LE NEUBOURG (FR)

(21) Appl. No.: 19/100,242

(22) PCT Filed: Aug. 2, 2023
(86) PCT No.: PCT/FR2023/051230

§ 371 Date: Jan. 31, 2025

(30) Foreign Application Priority Data
Aug. 3, 2022 (FR) FR2208054

(51) Int. Cl. B05B 11/10

(52) U.S. Cl. CPC B05B 11/1026; B05B 11/1047; B05B 11/1061; B05B 11/1074; B05B 11/1077

(57) ABSTRACT

A device for dispensing a fluid product having a dispensing head and a pump including a pump body having a pump chamber. The pump body has a lower opening closed by an inlet valve. A piston slides in the pump body around a hollow axial rod connected to the head, the rod provided with an inner channel closed on the lower side and having a radial hole. The piston has an outer sealing lip sealing against the pump body and an inner sealing lip sealing the radial hole when in the non-operating state. A resilient return member biases the pump towards its non-operating position after actuation. The valve holder has a sleeve, an upper edge of which mechanically abuts the piston during start-up to move the piston axially upwards to open the radial hole and discharge the air in the pump chamber.

The present invention relates to a device for dispensing a fluid product.

Documents FR2343137 and FR2403465 describe a device having a pump comprising a pump body in which a piston can move, slidingly mounted on an actuating rod, the thrust force exerted on the rod by the user being transmitted to the piston through a spring, such that, by actuating the actuating rod, a relative movement of the piston with respect to the rod occurs.

Document WO2020128269A1 describes an improved version of the pump above.

These pumps however have certain disadvantages. Thus, they comprise two metal springs, which is not advantageous from a recycling point of view. In addition, they comprise a gland, cooperating with the return spring and necessary to achieve the start-up of the pump.

The present invention aims to provide a device for dispensing a fluid product which does not reproduce the abovementioned disadvantages.

The present invention also aims to provide a device for dispensing a fluid product, which improves recycling by removing the metal springs.

Thus, the present invention also aims to provide a device for dispensing a fluid product which simplifies the start-up.

The present invention also aims to provide a device for dispensing a fluid product which is simple and inexpensive to manufacture and to assemble.

The present invention therefore aims for a device for dispensing a fluid product comprising a dispensing head provided with a dispensing hole, said dispensing head being assembled on a pump comprising:
a pump body, having a pump chamber, said pump body having an upper opening closed by a piston and a lower opening closed by an inlet valve, held in place by a valve holder mounted in said pump body,
said piston sealingly sliding in said pump body around a hollow axial rod connected to said dispensing head, said hollow axial rod being provided with an inner axial channel axially closed on the lower side and communicating with the outside by a radial hole, said piston having, on an outer radial edge, an outer sealing lip, configured to seal against said pump body, and on an inner radial edge, an inner sealing lip, configured to seal said radial hole when it is in the non-operating state,
a resilient return member to bias said pump towards its non-operating position, and to guide it back towards this non-operating position after each actuation,
said valve holder having an axial sleeve projecting axially upwards, an upper edge of which mechanically abuts with said piston, in particular said inner sealing lip, during start-up, to move said piston axially upwards with respect to said rod to open said radial hole and discharge the air contained in said pump chamber.

Advantageously, said resilient return member is non-metallic.

Advantageously, said resilient return member is a bellows disposed between said dispensing head and a part mounted on said pump body, such as a fixing ring.

Advantageously, a hollow axial insert is fixed in said hollow axial rod, said hollow axial insert having said radial hole.

Advantageously, a fixing ring is provided to fix said pump body on a reservoir with a neck seal interposed therebetween, a ring seal being provided for ensuring the sealing of said fixing ring with said pump body.

Advantageously, in the non-operating position, an upper axial edge of said piston sealingly engages with said ring seal.

Advantageously, a vent is provided in said pump body to allow the product expelled by the pump to be replaced by air on each actuation.

Advantageously, said piston comprises at least one deformable lip engaging during actuation with a frustoconical part of said hollow axial rod, to generate a precompression.

Advantageously, said upper edge of said axial sleeve comprises at least one opening, to enable air to pass during start-up.

Advantageously, each opening is formed by a notch made in said upper edge of said sleeve.

These features and advantages and others will appear more clearly during the detailed description below, made in reference to the accompanying drawings, given as non-limiting examples, and in which:

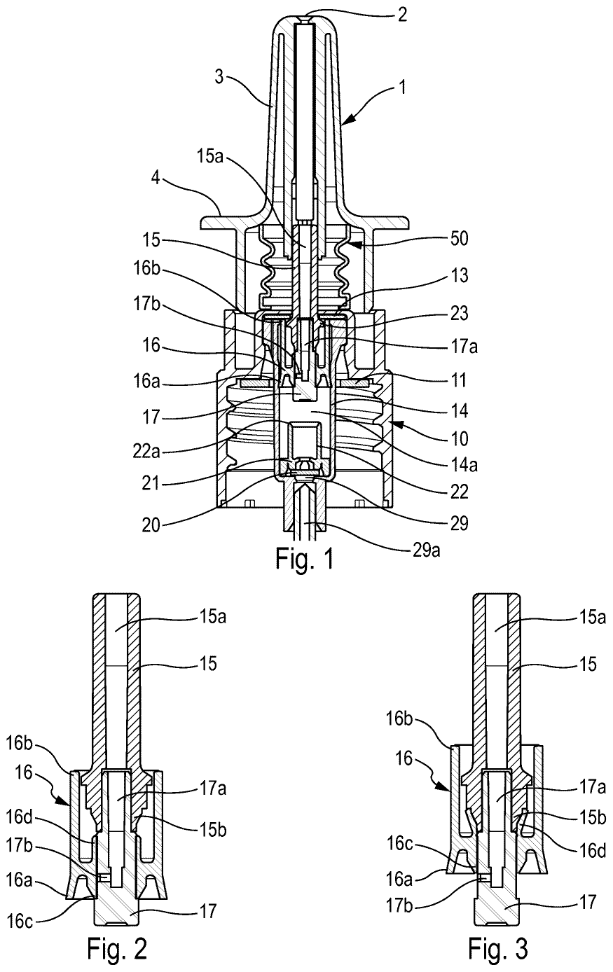

FIG. 1 is a schematic, cross-sectional view of a device for dispensing a fluid product comprising a pump for dispensing a fluid product according to a first advantageous embodiment of the invention, in the non-operating position,

FIGS. 2 and 3 are detailed, schematic, cross-sectional views showing the precompression, respectively before and during actuation, of the device of FIG. 1,

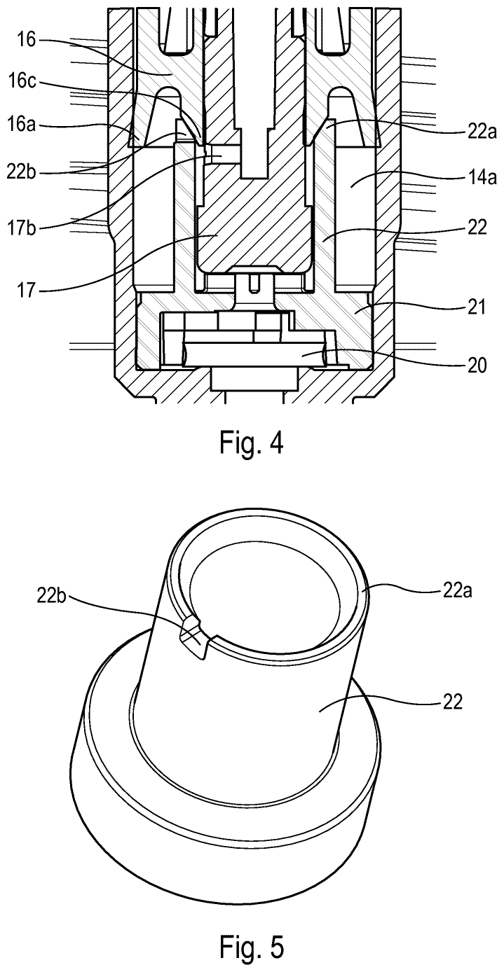

FIG. 4 is a detailed, schematic, cross-sectional view of a device for dispensing a fluid product comprising a pump for dispensing a fluid product according to a second advantageous embodiment of the invention, in the actuating position during start-up,

FIG. 5 is a detailed, schematic, perspective view of the valve holder of the device of FIG. 4.

In the description, the terms “upper”, “lower”, “top” and “bottom” refer to the upright position of the device represented in the figures. The terms “axial” and “radial” refer to the vertical central axis of the pump.

FIG. 1 represents a pump according to an advantageous embodiment of the invention, which improves a pump of the prior art such as described in document WO2020128269A1.

This pump comprises a pump body 14 containing a pump chamber 14a. The pump body 14 is axially open on both sides, with an upper opening closed by a piston 16 and a lower opening 29 closed by an inlet valve 20.

A fixing ring 10 is provided to fix the pump body 14 on the neck of a reservoir (not represented) with a neck seal 11 interposed therebetween. A ring seal 13 ensures the sealing of the fixing ring 10 with the pump body 14.

A piston 16 sealingly slides in the pump body 14, around a hollow axial rod 15, with a first inner axial channel 15a.

The first inner axial channel 15a opens axially towards the top of the rod 15 to connect to a dispensing head 1 provided with a dispensing hole 2.

The dispensing head 1 comprises a nasal tip 3 intended to be at least partially inserted into a nostril during use, as well as a radial flange 4 forming a support for the user's fingers during actuation.

Towards the bottom, the first inner axial channel 15a is also axially open and the hollow axial rod 15 receives a hollow axial insert 17.

This hollow axial insert 17 comprises a second inner axial channel 17a which opens axially upwards into the first inner axial channel 15a of the hollow axial rod 15.

Towards the bottom, the second inner axial channel 17a is axially closed, and the hollow axial insert comprises a radial hole 17b to connect the second inner axial channel 17a with the outside of the hollow axial insert 17.

Optionally, the rod 15 and the insert 17 could be formed of one single piece, for example by moulding.

A non-metallic resilient return member 50 is provided to bias the pump towards its non-operating position, and guide it back towards this non-operating position after each actuation.

In the example represented, this resilient return member 50 is formed by a bellows arranged between the dispensing head 1 and the fixing ring 10, which resiliently biases the dispensing head towards its non-operating position. During actuation, this bellows 50 is compressed, such that after actuation, the compressed bellows will resiliently return towards its non-compressed position, driving with it, the dispensing head 1 towards its non-operating position, which in turn, returns the piston 16 towards its non-operating position by suctioning the next dose of fluid product into the pump chamber 14a.

An outer radial edge of the piston 16 forms an outer sealing lip 16a, for ensuring the sealing with respect to the inner surface of the pump body 14.

An upper axial edge 16b of the piston 16 ensures, in the non-operating state, the sealing against the ring seal 13.

The pump body 14 is closed towards the bottom by the inlet valve 20, held in place by a valve holder 21. This inlet valve 20 closes the lower opening 29, communicating with the inside of the reservoir, and on which a dip tube 29a can be connected, in the well-known conventional manner.

The rod 15 comprises a shoulder 23, which serves to limit the upward movement of the rod 15, by forming an abutment with the ring seal 13.

When the pump is in the non-operating state, the upper edge 16b of the piston 16 is applied against the ring seal 13.

The sealing between the piston 16 and the hollow axial insert 17 is ensured by an inner part of the piston 16, preferably conically-shaped, forming an inner sealing lip 16c.

In the non-operating state, the radial hole 17b is in line with the inner sealing lip 16c, so as to be fully closed. The sealing between the outside and the inside of the reservoir is therefore ensured, on the one hand, by the contact between the upper edge 16b of the piston 16 and the ring seal 13, and on the other hand, between the inner sealing lip 16c and the hollow axial insert 17.

To achieve a precompression during actuation, which makes it possible, in particular, to ensure a good dispensing of the entire dose without risk of partial actuation stroke, the piston 16 can comprise at least one deformable lip 16d cooperating with a frustoconical part 15b of the hollow axial rod 15. FIGS. 2 and 3 illustrate this precompression. In a variant, the frustoconical part could also be made on the hollow axial insert 17.

In the non-operating state, the lip 16d is substantially non-deformed, and at the start of the actuation stroke, it will cooperate with the frustoconical part 15b which will progressively deform said lip 16d. This resilient deformation generates resistance to actuation, and therefore a precompression, which requires a certain threshold force to actuate the pump.

In addition, this resilient deformation of the lip 16d resiliently biases the piston 16 towards its non-operating position, in which the inner sealing lip 16c of the piston closes the radial hole 17b of the hollow axial insert 17. Thus, after each actuation, as soon as the user releases their pressure on the dispensing head 1, the piston 16 automatically returns to its position of closing the radial hole 17b, thus enabling the pump chamber 14a to be filled with another dose of fluid product suctioned from the reservoir.

A vent (not represented) can advantageously be provided in the pump body 14 to allow the product expelled by the pump 14 to be replaced by air on each actuation.

The pump described above operates as follows. In the non-operating state, the sealing inside the reservoir (not represented) is ensured by the neck seal 11 between the ring 10 and the reservoir (not represented), by the ring seal 13 between the ring 10 and the pump body 14, by the contact between the upper edge 16b of the piston 16 and the ring seal 13, and by the inner sealing lip 16c of the piston 16 against the hollow axial insert 17.

The reservoir, not represented in the drawings, and which may be any, is filled with a liquid, in particular a pharmaceutical liquid, to be dispensed, in particular to be sprayed.

The fixing ring 10 can be made of plastic material. It can be screwed, crimped, snap-fitted or otherwise fixed on the reservoir.

The user must first start up the pump by operating it to expel the air until the pump chamber 14a is filled with liquid. During start-up, the air contained in the pump chamber 14a is compressed, and it is by a mechanical abutment between the valve holder 21 and the piston 16 that the insert 17 and the rod 15 can move with respect to the piston 16 to open the radial hole 17b and discharge the compressed air. When the pump returns to the non-operating position, liquid is suctioned into the pump chamber 14a through the dip tube 29a, in a well-known manner.

According to the invention, the valve holder 21 comprises an axial sleeve 22 projecting axially upwards, the upper edge 22a of which cooperates during start-up with the piston 16, in particular the inner sealing lip 16c, to move the piston 16 axially upwards, to open the radial hole 17b and discharge the air contained in the pump chamber 14a.

Advantageously, as shown in the second embodiment of FIGS. 4 and 5, the upper edge 22a of the axial sleeve 22 can comprise one or more openings 22b, to enable the passage of air upon start-up. Thus, as can be seen in FIG. 4, the discharging of air is ensured even in case of contact over the entire periphery between the upper edge 22a of the sleeve and the inner sealing lip 16c of the piston. Advantageously, each opening 22b can be formed by a notch made in the upper edge 22a of the sleeve 22.

The present invention therefore makes it possible to achieve a reliable start-up without using an additional part, such as a gland, interspersed between the piston 16 and the rod 15 and/or the insert 17, as is the case in the pump of document WO2020128269A1.

After start-up, the pump is ready to be used to dispense a dose of liquid upon each actuation.

To perform such an actuation, the user presses axially on the dispensing head 1 to push in the rod 15. With the liquid being incompressible, the piston 16 moves around the insert 17 while deforming the deformable lip 16d on the frustoconical profile 15b of the rod 15. When the pressure is sufficient to overcome the resistance to deformation of the deformable lip 16d, the piston 16 uncovers the radial hole 17b, discharging the liquid out of the pump chamber 14a.

The liquid is rapidly discharged through the radial hole 17b, the second inner channel 17a and the first inner channel 15a, towards the dispensing head 1 assembled on the pump.

Thereafter, the user ceases to exert a pressure on the rod 15. The deformable lip 16d immediately biases the piston 16 to return to its non-operating position, which ensures the closing of the radial hole 17b, and closes the pump chamber 14a with respect to the dispensing head 1. At the same time, the bellows 50 returns the dispensing head 1 into its non-operating position, which drives the rod 15 and the insert 17 upwards. This creates a depression in the pump chamber 14a, such that liquid is suctioned through the hole 29, raising the valve 20, while air replaces this liquid in the reservoir, by passing through the central opening of the ring seal 13 around the rod 15, and through the vent 35. When the upper edge 16 b of the piston 16 comes into contact with the ring seal 13, the air can no longer enter, and the piston 16 is blocked, the pump has returned to its non-operating position and is ready for another actuation.

An advantage of the pump represented in FIG. 1 is that it comprises no metal parts, which on the one hand, makes the manufacture and assembly of the pump less complex and therefore less expensive, and which on the other hand, is advantageous for recycling.

Another advantage is that the pump ensures a reliable start-up without an intermediate part between the piston and the rod and/or the insert.

The present invention has been described with reference to an advantageous embodiment, but it is understood that a person skilled in the art can provide any modifications to this, without going beyond the scope of the present invention, such as defined by the accompanying claims.

1-10. (canceled)

11. A device for dispensing a fluid product comprising a dispensing head provided with a dispensing hole, said dispensing head being assembled on a pump comprising:
a pump body, having a pump chamber, said pump body having an upper opening closed by a piston and a lower opening closed by an inlet valve, held in place by a valve holder fixed in said pump body,
said piston sealingly sliding in said pump body around a hollow axial rod connected to said dispensing head, said hollow axial rod being provided with an inner axial channel axially closed on the lower side and communicating with the outside by a radial hole, said piston having, on an outer radial edge, an outer sealing lip configured to seal against said pump body, and on an inner radial edge, an inner sealing lip, configured to seal the radial hole when it is in the non-operating state,
an elastic return member to bias said pump towards its non-operating position, and to guide it back to this non-operating position after each actuation,
wherein said valve holder comprises an axial sleeve projecting axially upwards, an upper edge of which mechanically abuts with said inner sealing lip of said piston, during start-up, when said pump chamber contains air, in order to move said piston axially upwards with respect to said rod in order to open said radial hole and discharge the air contained in said pump chamber.

12. The device according to claim 11, wherein said dispensing member is non-metallic.

13. The device according to claim 12, wherein said resilient return member is a bellows disposed between said dispensing head and a part fixed on said pump body, such as a fixing ring.

14. The device according to claim 11, wherein a hollow axial insert is fixed in said hollow axial rod, said hollow axial insert having said radial hole.

15. The device according to claim 11, wherein a fixing ring is provided to fixer said pump body on a reservoir with a neck seal interposed therebetween, a ring seal being provided for ensuring the sealing of said fixing ring with said pump body.

16. The device according to claim 15, wherein, in the non-operating position, an upper axial edge of said piston sealingly cooperates with said ring seal.

17. The device according to claim 11, wherein a vent is provided in said pump body to allow the product expelled by the pump to be replaced by air on each actuation.

18. The device according to claim 11, wherein said piston comprises at least one deformable lip cooperating during actuation with a frustoconical part of said hollow axial rod to generate a precompression.

19. The device according to claim 11, wherein said upper edge of said axial sleeve includes at least one opening to enable the passage of air upon start-up.

20. The device according to claim 19, wherein each opening is formed by a notch made in said upper edge of said sleeve.
